SEQUENCE LISTING (24 sequences)

SEQ ID NO: 1 - DNA, 1428 bp, Artificial Sequence
  Synthetic
atggctatcattgacagcaagacaactctcccacgccactcactcatccacaccatcaagctcaactccaacaaaaagtatggccctggtgacatgacaaatgggaaccagttcatcatttccaagcaagagtgggcaaccattggtgcttacattcagactggattgggcttgccagtgaatgagcagcaattgaggactcacgtcaacctctcacaagacatcagcataccatctgacttttcccaactctatgatgtctactgttctgacaagacttcagcagaatggtggaacaagaatctctatcctttgattatcaagtctgccaatgacattgcttcttatggcttcaaggtggctggtgatccaagcatcaagaaagatggctacttcaagaaacttcaagatgaacttgacaacattgttgacaacaattctgatgacgatgcaatagccaaggccatcaaggacttcaaggcaaggtgtggcatactcatcaaggaggccaagcagtatgaagaggcagccaagaacattgtgacttcattggatcagttccttcatggagaccagaagaaacttgagggtgtcatcaacattcagaaacgtctcaaggaggttcaaacagctctcaatcaagcacatggggaatcctcaccagctcacaaagaactccttgagaaagtgaagaacttgaaaaccacacttgagaggaccatcaaagctgaacaagacttggaaaagaaagttgagtacagctttctccttggacctctccttggctttgttgtctatgagattcttgagaatactgctgttcaacacatcaagaatcagattgatgagatcaagaaacagttggattctgcccaacatgacttggatcgtgatgtgaagatcattgggatgctcaacagcatcaacactgacattgacaacttgtatagccaaggacaagaagccatcaaggtctttcagaagttgcaagggatatgggcaaccattggtgctcagatagagaatcttcgcacaacttcccttcaagaagtccaagattctgacgatgctgatgaaatacagattgaacttgaggatgcctctgatgcctggcttgttgtggctcaagaagccagagacttcacactcaatgcttactccaccaacagcagacagaatctccccatcaatgtgatctcagattcatgcaactgctccaccacaaacatgacttccaatcagtacagcaaccccaccacaaacatgaccagcaatcagtacatgattagccatgagtacacttcattgcccaacaatttcatgttgtccagaaactccaaccttgagtacaagtgccctgagaacaacttcatgatctactggtacaacaattctgactggtacaacaattctgactggtacaacaat

SEQ ID NO: 2 - protein, 476 aa, Artificial Sequence
  Derived
MAIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNN

SEQ ID NO: 3 - DNA, 1425 bp, Artificial Sequence
  Synthetic
atgatcattgactctaagaccactcttccacggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgaggaatagcaatctggagtacaagtgtccggagaacaacttcatgatctactggtacaacaactccgattggtacaacaactctgactggtacaacaac

SEQ ID NO: 4 - protein, 475 aa, Artificial Sequence
  Derived
MIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNN

SEQ ID NO: 5 - DNA, 1434 bp, Artificial Sequence
  Synthetic
atggctatcattgacagcaagacaactctcccacgccactcactcatccacaccatcaagctcaactccaacaaaaagtatggccctggtgacatgacaaatgggaaccagttcatcatttccaagcaagagtgggcaaccattggtgcttacattcagactggattgggcttgccagtgaatgagcagcaattgaggactcacgtcaacctctcacaagacatcagcataccatctgacttttcccaactctatgatgtctactgttctgacaagacttcagcagaatggtggaacaagaatctctatcctttgattatcaagtctgccaatgacattgcttcttatggcttcaaggtggctggtgatccaagcatcaagaaagatggctacttcaagaaacttcaagatgaacttgacaacattgttgacaacaattctgatgacgatgcaatagccaaggccatcaaggacttcaaggcaaggtgtggcatactcatcaaggaggccaagcagtatgaagaggcagccaagaacattgtgacttcattggatcagttccttcatggagaccagaagaaacttgagggtgtcatcaacattcagaaacgtctcaaggaggttcaaacagctctcaatcaagcacatggggaatcctcaccagctcacaaagaactccttgagaaagtgaagaacttgaaaaccacacttgagaggaccatcaaagctgaacaagacttggaaaagaaagttgagtacagctttctccttggacctctccttggctttgttgtctatgagattcttgagaatactgctgttcaacacatcaagaatcagattgatgagatcaagaaacagttggattctgcccaacatgacttggatcgtgatgtgaagatcattgggatgctcaacagcatcaacactgacattgacaacttgtatagccaaggacaagaagccatcaaggtctttcagaagttgcaagggatatgggcaaccattggtgctcagatagagaatcttcgcacaacttcccttcaagaagtccaagattctgacgatgctgatgaaatacagattgaacttgaggatgcctctgatgcctggcttgttgtggctcaagaagccagagacttcacactcaatgcttactccaccaacagcagacagaatctccccatcaatgtgatctcagattcatgcaactgctccaccacaaacatgacttccaatcagtacagcaaccccaccacaaacatgaccagcaatcagtacatgattagccatgagtacacttcattgcccaacaatttcatgttgtccagaaactccaaccttgagtacaagtgccctgagaacaacttcatgatctactggtacaacaattctgactggtacaacaattctgactggtacaacaatccacct

SEQ ID NO: 6 - protein, 478 aa, Artificial Sequence
  Derived
MAIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNNPP

SEQ ID NO: 7 - DNA, 1431 bp, Artificial Sequence
  Synthetic
atgatcattgactctaagaccactcttccacggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgaggaatagcaatctggagtacaagtgtccggagaacaacttcatgatctactggtacaacaactccgattggtacaacaactctgactggtacaacaacccacct

SEQ ID NO: 8 - protein, 477 aa, Artificial Sequence
  Derived
MIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNNPP

SEQ ID NO: 9 - DNA, 1305 bp, Artificial Sequence
  Synthetic
atgggtcgccactcactcatccacaccatcaagctcaactccaacaaaaagtatggccctggtgacatgacaaatgggaaccagttcatcatttccaagcaagagtgggcaaccattggtgcttacattcagactggattgggcttgccagtgaatgagcagcaattgaggactcacgtcaacctctcacaagacatcagcataccatctgacttttcccaactctatgatgtctactgttctgacaagacttcagcagaatggtggaacaagaatctctatcctttgattatcaagtctgccaatgacattgcttcttatggcttcaaggtggctggtgatccaagcatcaagaaagatggctacttcaagaaacttcaagatgaacttgacaacattgttgacaacaattctgatgacgatgcaatagccaaggccatcaaggacttcaaggcaaggtgtggcatactcatcaaggaggccaagcagtatgaagaggcagccaagaacattgtgacttcattggatcagttccttcatggagaccagaagaaacttgagggtgtcatcaacattcagaaacgtctcaaggaggttcaaacagctctcaatcaagcacatggggaatcctcaccagctcacaaagaactccttgagaaagtgaagaacttgaaaaccacacttgagaggaccatcaaagctgaacaagacttggaaaagaaagttgagtacagctttctccttggacctctccttggctttgttgtctatgagattcttgagaatactgctgttcaacacatcaagaatcagattgatgagatcaagaaacagttggattctgcccaacatgacttggatcgtgatgtgaagatcattgggatgctcaacagcatcaacactgacattgacaacttgtatagccaaggacaagaagccatcaaggtctttcagaagttgcaagggatatgggcaaccattggtgctcagatagagaatcttcgcacaacttcccttcaagaagtccaagattctgacgatgctgatgaaatacagattgaacttgaggatgcctctgatgcctggcttgttgtggctcaagaagccagagacttcacactcaatgcttactccaccaacagcagacagaatctccccatcaatgtgatctcagattcatgcaactgctccaccacaaacatgacttccaatcagtacagcaaccccaccacaaacatgaccagcaatcagtacatgattagccatgagtacacttcattgcccaacaatttcatgttgtccaga

SEQ ID NO: 10 - DNA, 1305 bp, Artificial Sequence
  Synthetic
atgggccggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgagg

SEQ ID NO: 11 - protein, 435 aa, Artificial Sequence
  Derived
MGRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSR

SEQ ID NO: 12 - DNA, 1311 bp, Artificial Sequence
  Synthetic
atgggtcgccactcactcatccacaccatcaagctcaactccaacaaaaagtatggccctggtgacatgacaaatgggaaccagttcatcatttccaagcaagagtgggcaaccattggtgcttacattcagactggattgggcttgccagtgaatgagcagcaattgaggactcacgtcaacctctcacaagacatcagcataccatctgacttttcccaactctatgatgtctactgttctgacaagacttcagcagaatggtggaacaagaatctctatcctttgattatcaagtctgccaatgacattgcttcttatggcttcaaggtggctggtgatccaagcatcaagaaagatggctacttcaagaaacttcaagatgaacttgacaacattgttgacaacaattctgatgacgatgcaatagccaaggccatcaaggacttcaaggcaaggtgtggcatactcatcaaggaggccaagcagtatgaagaggcagccaagaacattgtgacttcattggatcagttccttcatggagaccagaagaaacttgagggtgtcatcaacattcagaaacgtctcaaggaggttcaaacagctctcaatcaagcacatggggaatcctcaccagctcacaaagaactccttgagaaagtgaagaacttgaaaaccacacttgagaggaccatcaaagctgaacaagacttggaaaagaaagttgagtacagctttctccttggacctctccttggctttgttgtctatgagattcttgagaatactgctgttcaacacatcaagaatcagattgatgagatcaagaaacagttggattctgcccaacatgacttggatcgtgatgtgaagatcattgggatgctcaacagcatcaacactgacattgacaacttgtatagccaaggacaagaagccatcaaggtctttcagaagttgcaagggatatgggcaaccattggtgctcagatagagaatcttcgcacaacttcccttcaagaagtccaagattctgacgatgctgatgaaatacagattgaacttgaggatgcctctgatgcctggcttgttgtggctcaagaagccagagacttcacactcaatgcttactccaccaacagcagacagaatctccccatcaatgtgatctcagattcatgcaactgctccaccacaaacatgacttccaatcagtacagcaaccccaccacaaacatgaccagcaatcagtacatgattagccatgagtacacttcattgcccaacaatttcatgttgtccagaccacct

SEQ ID NO: 13 - DNA, 1311 bp, Artificial Sequence
  Synthetic
atgggccggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgaggccacct

SEQ ID NO: 14 - protein, 437 aa, Artificial Sequence
  Derived
MGRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRPP

SEQ ID NO: 15 - DNA, 1512 bp, Artificial Sequence
  Synthetic
atggctcgtgcacaacttgtccttgtggcattggttgcagctgctctgctcttggctggtcctcacaccacaatggctatcattgacagcaagacaactctcccacgccactcactcatccacaccatcaagctcaactccaacaaaaagtatggccctggtgacatgacaaatgggaaccagttcatcatttccaagcaagagtgggcaaccattggtgcttacattcagactggattgggcttgccagtgaatgagcagcaattgaggactcacgtcaacctctcacaagacatcagcataccatctgacttttcccaactctatgatgtctactgttctgacaagacttcagcagaatggtggaacaagaatctctatcctttgattatcaagtctgccaatgacattgcttcttatggcttcaaggtggctggtgatccaagcatcaagaaagatggctacttcaagaaacttcaagatgaacttgacaacattgttgacaacaattctgatgacgatgcaatagccaaggccatcaaggacttcaaggcaaggtgtggcatactcatcaaggaggccaagcagtatgaagaggcagccaagaacattgtgacttcattggatcagttccttcatggagaccagaagaaacttgagggtgtcatcaacattcagaaacgtctcaaggaggttcaaacagctctcaatcaagcacatggggaatcctcaccagctcacaaagaactccttgagaaagtgaagaacttgaaaaccacacttgagaggaccatcaaagctgaacaagacttggaaaagaaagttgagtacagctttctccttggacctctccttggctttgttgtctatgagattcttgagaatactgctgttcaacacatcaagaatcagattgatgagatcaagaaacagttggattctgcccaacatgacttggatcgtgatgtgaagatcattgggatgctcaacagcatcaacactgacattgacaacttgtatagccaaggacaagaagccatcaaggtctttcagaagttgcaagggatatgggcaaccattggtgctcagatagagaatcttcgcacaacttcccttcaagaagtccaagattctgacgatgctgatgaaatacagattgaacttgaggatgcctctgatgcctggcttgttgtggctcaagaagccagagacttcacactcaatgcttactccaccaacagcagacagaatctccccatcaatgtgatctcagattcatgcaactgctccaccacaaacatgacttccaatcagtacagcaaccccaccacaaacatgaccagcaatcagtacatgattagccatgagtacacttcattgcccaacaatttcatgttgtccagaaactccaaccttgagtacaagtgccctgagaacaacttcatgatctactggtacaacaattctgactggtacaacaattctgactggtacaacaataaggacgagttg

SEQ ID NO: 16 - protein, 504 aa, Artificial Sequence
  Derived
MARAQLVLVALVAAALLLAGPHTTMAIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNNKDEL

SEQ ID NO: 17 - DNA, 1509 bp, Artificial Sequence
  Synthetic
atggctcgtgcacaacttgtccttgtggcattggttgcagctgctctgctcttggctggtcctcacaccacaatgatcattgactctaagaccactcttccacggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgaggaatagcaatctggagtacaagtgtccggagaacaacttcatgatctactggtacaacaactccgattggtacaacaactctgactggtacaacaacaaggacgagttg

SEQ ID NO: 18 - protein, 503 aa, Artificial Sequence
  Derived
MARAQLVLVALVAAALLLAGPHTTMIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNNKDEL

SEQ ID NO: 19 - DNA, 1416 bp, Artificial Sequence
  Synthetic
atggctcgtgcacaacttgtccttgtggcattggttgcagctgctctgctcttggctggtcctcacaccacaatggctatcattgacagcaagacaactctcccacgccactcactcatccacaccatcaagctcaactccaacaaaaagtatggccctggtgacatgacaaatgggaaccagttcatcatttccaagcaagagtgggcaaccattggtgcttacattcagactggattgggcttgccagtgaatgagcagcaattgaggactcacgtcaacctctcacaagacatcagcataccatctgacttttcccaactctatgatgtctactgttctgacaagacttcagcagaatggtggaacaagaatctctatcctttgattatcaagtctgccaatgacattgcttcttatggcttcaaggtggctggtgatccaagcatcaagaaagatggctacttcaagaaacttcaagatgaacttgacaacattgttgacaacaattctgatgacgatgcaatagccaaggccatcaaggacttcaaggcaaggtgtggcatactcatcaaggaggccaagcagtatgaagaggcagccaagaacattgtgacttcattggatcagttccttcatggagaccagaagaaacttgagggtgtcatcaacattcagaaacgtctcaaggaggttcaaacagctctcaatcaagcacatggggaatcctcaccagctcacaaagaactccttgagaaagtgaagaacttgaaaaccacacttgagaggaccatcaaagctgaacaagacttggaaaagaaagttgagtacagctttctccttggacctctccttggctttgttgtctatgagattcttgagaatactgctgttcaacacatcaagaatcagattgatgagatcaagaaacagttggattctgcccaacatgacttggatcgtgatgtgaagatcattgggatgctcaacagcatcaacactgacattgacaacttgtatagccaaggacaagaagccatcaaggtctttcagaagttgcaagggatatgggcaaccattggtgctcagatagagaatcttcgcacaacttcccttcaagaagtccaagattctgacgatgctgatgaaatacagattgaacttgaggatgcctctgatgcctggcttgttgtggctcaagaagccagagacttcacactcaatgcttactccaccaacagcagacagaatctccccatcaatgtgatctcagattcatgcaactgctccaccacaaacatgacttccaatcagtacagcaaccccaccacaaacatgaccagcaatcagtacatgattagccatgagtacacttcattgcccaacaatttcatgttgtccagaaaggacgagttg

SEQ ID NO: 20 - protein, 472 aa, Artificial Sequence
  Derived
MARAQLVLVALVAAALLLAGPHTTMAIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRKDEL

SEQ ID NO: 21 - DNA, 1413 bp, Artificial Sequence
  Synthetic
atggctcgtgcacaacttgtccttgtggcattggttgcagctgctctgctcttggctggtcctcacaccacaatgatcattgactctaagaccactcttccacggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgaggaaggacgagttg

SEQ ID NO: 22 - protein, 471 aa, Artificial Sequence
  Derived
MARAQLVLVALVAAALLLAGPHTTMIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRKDEL

SEQ ID NO: 23 - DNA, 1329 bp, Artificial Sequence
  Synthetic
atgatcattgactctaagaccactcttccacggcacagcttgatacacactatcaagttgaactcgaacaagaagtatggacctggtgacatgaccaacggcaatcagttcatcatttcaaagcaagaatgggctacaataggtgcgtacattcagactgggctgggactcccagtgaacgaacaacaactgaggacccacgtcaatctcagccaagacatttcaatcccctcagactttagccagctctacgacgtttactgctccgacaagacctcggctgagtggtggaacaagaacctctatcctctcatcatcaaatcagcaaatgacatagcctcctatggcttcaaggttgctggggacccgtccatcaagaaagatggatacttcaagaagctccaagacgagcttgataacattgttgataacaattccgatgatgacgccatcgcgaaggccatcaaagacttcaaagccagatgtgggattctgatcaaggaggcgaagcagtacgaggaagctgcgaagaacatagtgacgtccttggaccagttcttgcatggcgaccagaagaagttggaaggggtgatcaacattcagaaaaggctcaaagaggttcagacagcgctcaaccaagcacacggagaaagctcaccagcccacaaggaacttctggagaaggtgaagaatcttaagaccactcttgagcgcacgatcaaggctgagcaagatttggagaagaaagtcgagtacagcttccttctgggtcctttgctgggctttgtggtgtacgagatcctcgaaaacacggctgtgcagcacatcaagaatcagatcgacgagatcaagaagcaacttgactctgctcagcatgaccttgacagagatgtgaagatcatagggatgctcaattcgatcaacactgatatcgacaatctgtattcacaaggccaagaagcgatcaaggtctttcagaaactgcaaggcatctgggcaacgattggtgctcagatcgagaaccttaggaccacctcgctgcaagaggtccaagactccgatgatgcggatgagatccagattgagttggaggatgccagcgacgcatggctggttgttgcccaagaggcacgggacttcacgttgaacgcctatagcacgaactctcgccagaatctgcccatcaacgtcatctcagactcgtgcaactgctctacaactaacatgacgagcaatcagtacagcaatcccaccacaaacatgacctccaatcagtacatgatctctcatgagtacacatcgctgccgaacaacttcatgctgtcgagg

SEQ ID NO: 24 - protein, 443 aa, Artificial Sequence
  Derived
MIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSR

(19) United States
(12) Patent Application Publication
(10) Pub. No.: US 2011/0225681 A1
(43) Pub. Date: Sep. 15, 2011

(54) Modified Bacillus Thuringiensis Cry6 Proteins For Nematode Control

(75) Inventors: Timohty D. Hey, Zionsville, IN (US); Kenneth Narva, Zionsville, IN (US); Aaron T. Woosley, Fishers, IN (US)

(73) Assignee: Dow AgroSciences LLC, Indianapolis, IN (US)

(21) Appl. No.: 13/060,242

(22) PCT Filed: Aug. 25, 2009
(86) PCT No.: PCT/US09/54927

§ 371 Date: Jun. 1, 2011

Related U.S. Application Data
(60) Provisional application No. 61/091,666, filed on Aug. 25, 2008.

(51) Int. Cl. A01H 5/00; C07H 21/04; C07K 14/325; C12N 5/10; A01N 37/18; A01P 5/00

(52) U.S. Cl. 800/301; 536/23.7; 530/350; 435/418; 514/4.6

(57) ABSTRACT

The subject invention concerns plants protected from nematode feeding damage and improved versions of Cry proteins. The subject invention also concerns improved versions of Cry6A proteins. Synthetic genes encoding these modified proteins are also part of the subject invention. Another embodiment of the subject invention includes plants transformed with the genes of the subject invention. In yet another embodiment the subject invention concerns Bt proteins for in-plant protection against crop damage by root knot nematode (RKN; Meloidogyne incognita) and soybean cyst nematode (SCN; Heterodera glycines).

### BACKGROUND OF THE INVENTION

Plant parasitic nematodes cause an adjusted economic loss of approximately $10 billion in the United States of America and $125 billion globally due to crop damage (Sasser and Freckman 1987; Chitwood 2003). Various nematode control strategies including chemicals are available to growers, but these management tools have drawbacks in terms of efficacy, expense and environmental safety. For example, methyl bromide, one of the main chemicals used to control plant parasitic nematodes, is being phased out due to environmental and human health concerns (Ristaino and Thomas 1997). There is therefore a need for improved nematode control technology with better pest efficacy and safety profiles.

Bacillus thuringiensis (Bt) and Bt insecticidal Cry proteins have a long history of safe use as biocontrol agents for crop protection (Betz et al., 2000). Bt proteins have been successfully used to control a variety of lepidopteran, coleopteran and dipteran insect pests, both as sprayable bioinsecticides and as plant-incorporated pesticides (Schnepf et al., 1998). Cry proteins are oral intoxicants that function by acting on midgut cells of susceptible insects. Classical three-domain insecticidal Bt proteins require activation as a first step in the intoxication of susceptible insects. Insecticidal Cry protein activation requires proteolytic removal of N-terminal and C-terminal regions (Bravo et al., 2007).

Compared to insecticidal Bts, less work has been conducted on the use of Bts for nematode control. Early studies reported the effects of Bt proteins on the viability of nematode eggs (Bottjer et al., 1985; Bone et al., 1985; Bone et al., 1987 Bone et al., 1988). Genes encoding several nematicidal Bt proteins have been cloned and expressed, and the encoded proteins have been demonstrated to have lethal effects on the free living nematode, Caenorhabditis elegans as described, for example, in U.S. Pat. Nos. 5,616,495; 6,632,792; 5,753,492; and U.S. Pat. No. 5,589,382. Nematicidal Cry proteins described in these patents include members of the Cry5, Cry6, Cry12, Cry13, Cry14, and Cry21 subfamilies. Nematicidal activity of some of these proteins has been demonstrated against a wider range of free-living nematodes (Wei et al., 2003). Further, Cry6Aa (U.S. Pat. No. 6,632,792) has been expressed in a tomato hairy root model system and shown to provide partial resistance to damage by the root knot nematode, Meloidogyne incognita (WO 2007/062064(A2); Li et al., 2007). However, to date, there has been no demonstration of Cry protein-mediated protection to nematode damage in stably transformed plants.

### BRIEF SUMMARY OF THE INVENTION

The subject invention concerns improved versions of Cry6Aa proteins. Synthetic genes encoding these modified proteins are also part of the subject invention. Another embodiment of the subject invention includes plants transformed with the genes of the subject invention. In yet another embodiment the subject invention concerns Bt proteins for in-plant protection against crop damage by root knot nematode (RKN; Meloidogyne species) and soybean cyst nematode (SCN; Heterodera glycines).

### BRIEF DESCRIPTION OF THE SEQUENCES

Some constructions required that two protein sequences be provided for Cry6A (dicot codon-optimized and maize codon-optimized). Thus, there are some differences between Cry6A protein sequences encoded by dicot and maize versions (those constructions noted). The sequences summarized below are polynucleotide/DNA sequences unless otherwise indicated to be protein/amino acid sequences.

SEQ ID NO:1 Cry6A Full Length (Dicot)

SEQ ID NO:2 Cry6A Full Length (Dicot) (Protein)

SEQ ID NO:3 Cry6A Full Length (Maize)

SEQ ID NO:4 Cry6A Full Length (Maize) (Protein)

SEQ ID NO:5 Cry6A Full Length+C-ter PP (Dicot)

SEQ ID NO:6 Cry6A Full Length+C-ter PP (Dicot) (Protein)

SEQ ID NO:7 Cry6A Full Length+C-ter PP (Maize)

SEQ ID NO:8 Cry6A Full Length+C-ter PP (Maize) (Protein)

SEQ ID NO:9 Cry6A N-ter+C-ter truncations (Dicot)

SEQ ID NO:10 Cry6A N-ter+C-ter truncations (Maize)

SEQ ID NO:11 Cry6A N-ter+C-ter truncations (Protein)

SEQ ID NO:12 Cry6A N-ter+C-ter truncations+C-ter PP (Dicot)

SEQ ID NO:13 Cry6A N-ter+C-ter truncations+C-ter PP (Maize)

SEQ ID NO:14 Cry6A N-ter+C-ter truncations+PP (Protein)

SEQ ID NO:15 Cry6A Full Length+ER signals (includes KDEL) (Dicot)

SEQ ID NO:16 Cry6A Full Length+ER signals (includes KDEL) (Dicot) (Protein)

SEQ ID NO:17 Cry6A Full Length+ER signals (includes KDEL) (Maize)

SEQ ID NO:18 Cry6A Full Length+ER signals (includes KDEL) (Maize) (Protein)

SEQ ID NO:19 Cry6A C-ter truncation+ER signals (includes KDEL) (Dicot)

SEQ ID NO:20 Cry6A C-ter truncation+ER signals (includes KDEL) (Dicot) (Protein)

SEQ ID NO:21 Cry6A C-ter truncations+ER signals (includes KDEL) (Maize)

SEQ ID NO:22 Cry6A C-ter truncation+ER signals (includes KDEL) (Maize) (Protein)

SEQ ID NO:23 DIG-264 Cry6A C-ter truncation (Maize)

SEQ ID NO:24 DIG-264 Cry6A C-ter truncation (Maize) (Protein)

### DETAILED DISCLOSURE OF THE INVENTION

The subject invention relates in part to protection of plants from damage by nematodes by the production in transgenic plants of certain nematode active Cry proteins. It is a further feature of the invention to disclose improvements to Cry protein efficacy made by engineering expression of the activated form of nematode-active Cry proteins. These modified Cry proteins are designed to have improved activity on plant parasitic nematodes including, but not limited to, root knot nematode (Meloidogyne species) and soybean cyst nematode (Heterodera glycines). Plant species which may be protected from nematode damage by the production of Cry proteins in transgenic varieties include, but are not limited to, corn, cotton, soybean, turf grasses, tobacco, sugar cane, sugar beets, citrus, peanuts, nursery stock, strawberries, vegetable crops, and bananas.

More specifically, the subject invention relates in part to surprisingly successful, improved Cry proteins designed to have N-terminal deletions and C-terminal deletions, either alone or in combination. Modified versions of Cry6Aa are described.

Cry6Aa has a unique predicted protein structure not related to three-domain Bt proteins. Modified versions of Cry6Aa are described that remove N-terminal and/or C-terminal sequences to yield protein variants that are not dependent on protease activation. These modified Cry6Aa variants have improved nematicidal activity. Additional modifications to some nematicidal proteins include addition of a carboxyl terminal proline-proline dipeptide to stabilize the protein (U.S. Pat. No. 7,122,516).

Further modifications and amino acid changes (including further deletions) can be made to proteins of the subject invention. The subject invention includes Cry6 proteins (with toxin activity), Cry6A proteins, and Cry6Aa proteins with such modifications. As used herein, the boundaries represent approximately 95% (Cry6Aa′ s), 78% (Cry6A′ s), and 45% (Cry6's) sequence identity per “Revision of the Nomenclature for the Bacillus thuringiensis Pesticidal Crystal Proteins,” N. Crickmore, D. R. Zeigler, J. Feitelson, E. Schnepf, J. Van Rie, D. Lereclus, J. Baum, and D. H. Dean. Microbiology and Molecular Biology Reviews (1998) Vol 62: 807-813. Proteins having at least 85% homology, and those having at least 90% homology to the subject Cry6 proteins can also be included within the scope of the subject invention.

Variants may be made by making random mutations or the variants may be designed. In the case of designed mutants, there is a high probability of generating variants with similar activity to the native toxin when amino acid identity is maintained in critical regions of the toxin which account for biological activity or are involved in the determination of three-dimensional configuration which ultimately is responsible for the biological activity. A high probability of retaining activity will also occur if substitutions are conservative. Amino acids may be placed in the following classes: non-polar, uncharged polar, basic, and acidic. Conservative substitutions whereby an amino acid of one class is replaced with another amino acid of the same type are least likely to materially alter the biological activity of the variant. Table 1 provides a listing of examples of amino acids belonging to each class.

TABLE 1

| Class of Amino Acid | Examples of Amino Acids |
|---|---|
| Nonpolar Side Chains | Ala, Val, Leu, Ile, Pro, Met, Phe, Trp |
| Uncharged Polar Side Chains | Gly, Ser, Thr, Cys, Tyr, Asn, Gln |
| Acidic Side Chains | Asp, Glu |
| Basic Side Chains | Lys, Arg, His |
| Beta-branched Side Chains | Thr, Val, Ile |
| Aromatic Side Chains | Tyr, Phe, Trp, His |

In some instances, non-conservative substitutions can also be made. The critical factor is that these substitutions must not significantly detract from the biological activity of the toxin. Variants include polypeptides that differ in amino acid sequence due to mutagenesis. Variant proteins encompassed by the present invention are biologically active, that is they continue to possess the desired biological activity of the native protein, that is, retaining pesticidal activity. Polynucleotides that hybridize with an exemplified or suggested sequence can be within the scope of the subject invention. Hybridization conditions include 1×SSPE and 42° C. or 65° C. See e.g. Keller, G. H., M. M. Manak (1987) DNA Probes, Stockton Press, New York, N.Y., pp. 169-170.

Genes encoding the improved Cry proteins described herein can be made by a variety of methods well-known in the art. For example, synthetic genes and synthetic gene segments can be made by phosphite tri-ester and phosphoramidite chemistry (Caruthers et al., 1987). Genes can be assembled in a variety of ways including, for example, by ligation of restriction fragments or polymerase chain reaction assembly of overlapping oligonucleotides (Stewart and Burgin, 2005). Further, terminal gene deletions can be made by PCR amplification using site-specific terminal oligonucleotides.

It should be noted that one skilled in the art, having the benefit of the subject disclosure, will recognize that the subject proteins can kill the target nematodes (and/or insects). Complete lethality, however, is not required. One preferred goal is to prevent nematodes/insects from damaging plants. Thus, prevention of feeding is sufficient, and “inhibiting” the nematodes/insects is likewise sufficient. This can be accomplished by making the nematodes/insects “sick” or by otherwise inhibiting (including killing) them so that damage to the plants being protected is reduced. Proteins of the subject invention can be used alone or in combination with another toxin (and/or other toxins) to achieve this inhibitory effect, which can also be referred to as “toxin activity.” Thus, the inhibitory function of the subject peptides can be achieved by any mechanism of action, directly or indirectly.

All patents, patent applications, provisional applications, and publications referred to or cited herein are incorporated by reference in their entirety to the extent they are not inconsistent with the explicit teachings of this specification.

Unless specifically indicated or implied, the terms “a”, “an”, and “the” signify “at least one” as used herein.

Following are examples that illustrate procedures for practicing the invention. These examples should not be construed as limiting. All percentages are by weight and all solvent mixture proportions are by volume unless otherwise noted. All temperatures are in degrees Celsius.

### Example 1

### Construction of Plant Expression Vectors Containing Genes Encoding Modified Cry6A Proteins

Cry6A full-length toxin coding regions were synthesized using commercial DNA synthesis vendors. Two versions of each coding region were constructed: one with a dicot codon bias, the other with a maize codon bias. Guidance regarding the design and production of synthetic genes can be found in, for example, WO 97/13402 and U.S. Pat. No. 5,380,831. In addition to the full length versions, several other gene versions were constructed, which encode novel Cry protein toxins. These included addition of a carboxyl terminal proline-proline dipeptide to stabilize the protein. Other modifications include truncations at the amino and carboxyl termini to create smaller toxins, which do not required proteolytic processing. Lastly, a series of toxins were made with endoplasmic reticulum targeting and retention signals.

All the modifications described above occur at the termini of the coding regions and represent either additions or deletions from either the 5′ and/or 3′ ends. These types of modification were done using sequence-specific primers and PCR amplification of gene products. The amplified products were subcloned into standard PCR product capture vectors and sequenced. The coding regions for the full-length and variant Cry6A proteins were then subcloned into plant transformation vectors containing the appropriate plant expression elements, thus producing binary vector plasmids such as pDAB7604 (comprising SEQ ID NO:1 which encodes SEQ ID NO:2), pDAB7565 (comprising SEQ ID NO:5 which encodes SEQ ID NO:6), pDAB7567 (comprising SEQ ID NO:9 which encodes SEQ ID NO:11), pDAB7569 (comprising SEQ ID NO:12 which encodes SEQ ID NO:14), pDAB7571 (comprising SEQ ID NO:15 which encodes SEQ ID NO:16), and pDAB7573 (comprising SEQ ID NO:19 which encodes SEQ ID NO:20), all of which may be used for the transformation of dicot plant species. The completed plant transformation vectors were used to transform a variety of plants as described below. Preferred constructs for the full-length and variant Cry6A proteins are: CsVMV v2 (promoter)—Cry coding region—Atu ORF24 3′ UTR (for dicots), and ZmUbil v2 (promoter)—Cry coding region—ZmPer5 3′ UTR v1 (for monocots). A preferred plant-expressible selectable marker gene comprises the DSM2 coding region flanked by appropriate plant transcriptional control elements. A second preferred plant-expressible selectable marker gene comprises the AAD1 coding region flanked by appropriate plant transcriptional control elements.

### Example 2

### Transformation of Arabidopsis

One aspect of the subject invention is the transformation of plants with genes encoding the nematicidal protein. The transformed plants are resistant to attack by the target pest.

Genes encoding modified Cry proteins, as disclosed herein, can be inserted into plant cells using a variety of techniques which are well known in the art. For example, a large number of cloning vectors comprising a replication system in E. coli and a marker that permits selection of the transformed cells are available for preparation for the insertion of foreign genes into higher plants. The vectors comprise, for example, pBR322, pUC series, M13 mp series, pACYC184, inter alia. Accordingly, the DNA fragment having the sequence encoding the modified Cry protein can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted.

The use of T-DNA for the transformation of plant cells has been intensively researched and sufficiently described in EP 120 516, Hoekema (1985), Fraley et al., (1986), and An et al., (1985).

Once the inserted DNA has been integrated in the plant genome, it is relatively stable. The transformation vector normally contains a selectable marker that confers on the transformed plant cells resistance to a biocide or an antibiotic, such as Bialaphos, Kanamycin, G418, Bleomycin, or Hygromycin, inter alia. The individually employed marker should accordingly permit the selection of transformed cells rather than cells that do not contain the inserted DNA.

A large number of techniques are available for inserting DNA into a plant host cell. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the right and left T-DNA border regions. They can be transformed directly into Agrobacteria (Holsters et al., 1978). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The transformed cells grow inside the plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties.

In a preferred embodiment of the subject invention, plants will be transformed with genes wherein the codon usage has been optimized for plants. See, for example, U.S. Pat. No. 5,380,831, which is hereby incorporated by reference. While some truncated toxins are exemplified herein, it is well-known in the Bt art that 130 kDa-type (full-length) toxins have an N-terminal half that is the core toxin, and a C-terminal half that is the protoxin “tail.” Thus, appropriate “tails” can be used with truncated/core toxins of the subject invention. See e.g. U.S. Pat. No. 6,218,188 and U.S. Pat. No. 6,673,990. In addition, methods for creating synthetic Bt genes for use in plants are known in the art (Stewart and Burgin, 2007).

Agrobacterium Transformation Standard cloning methods [as described in, for example, Sambrook et al., (1989) and Ausubel et al., (1995), and updates thereof] are used in the construction of binary plant expression plasmids. Restriction endonucleases are obtained from New England BioLabs (NEB; Beverly, Mass.), and T4 DNA Ligase (NEB Cat# M0202T) is used for DNA ligation. Plasmid preparations are performed using the Nucleospin Plasmid Preparation kit (Machery Nagel, Cat# 740 588.250) or the Nucleobond AX Xtra Midi kit (Machery Nagel, Cat# 740 410.100), following the instructions of the manufacturers. DNA fragments are purified using the QIAquick PCR Purification Kit (Qiagen, Valencia, Calif.; Cat# 28104) or the QIAEX II Gel Extraction Kit (Qiagen, Cat# 20021) after gel isolation.

The basic cloning strategy is to subclone full length and the modified Cry coding sequences (CDS) into pDAB8863 at the Nco I and Sac I restriction sites. The resulting plasmids are subcloned into the binary plasmid, pDAB3776, utilizing Gateway® technology. LR Clonase™ (Invitrogen, Carlsbad, Calif.; Cat# 11791-019) is used to recombine the full length and modified gene cassettes into the binary expression plasmid.

Electro-competent Agrobacterium tumefaciens (strain Z707S) cells are prepared and transformed using electroporation (Weigel and Glazebrook, 2002). 50 μL of competent Agrobacterium cells are thawed on ice and 10-25 ng of the desired plasmid is added to the cells. The DNA and cell mix is added to pre-chilled electroporation cuvettes (2 mm). An Eppendorf Electroporator 2510 is used for the transformation with the following conditions: Voltage: 2.4 kV, Pulse length: 5 msec. After electroporation, 1 mL of YEP broth is added to the cuvette and the cell-YEP suspension is transferred to a 15 mL culture tube. The cells are incubated at 28° in a water bath with constant agitation for 4 hours. After incubation, the culture is plated on YEP+agar with Erythromycin (200 mg/L) and Streptomycin (Sigma Chemical Co., St. Louis, Mo.) (250 mg/L). The plates are incubated for 2-4 days at 28°. Colonies are selected and streaked onto fresh YEP+agar with Erythromycin (200 mg/L) and Streptomycin (250 mg/L) plates and incubated at 28° for 1-3 days.

Colonies are selected for PCR analysis to verify the presence of the gene insert by using vector specific primers. Qiagen Spin Mini Preps, performed per manufacturer's instructions, are used to purify the plasmid DNA from selected Agrobacterium colonies with the following exception: 4 mL aliquots of a 15 mL overnight mini prep culture (liquid YEP+Spectinomycin (200 mg/L) and Streptomycin (250 mg/L)) are used for the DNA purification. Plasmid DNA from the binary vector used in the Agrobacterium transformation is included as a control. The PCR reaction is completed using Taq DNA polymerase from Invitrogen per manufacture's instructions at 0.5× concentrations. PCR reactions are carried out in a MJ Research Peltier Thermal Cycler programmed with the following conditions; 1) 94° for 3 minutes; 2) 94° for 45 seconds; 3) 55° for 30 seconds; 4) 72° for 1 minute per kb of expected product length; 5) 29 times to step 2; 6) 72° for 10 minutes. The reaction is maintained at 4° after cycling. The amplification is analyzed by 1% agarose gel electrophoresis and visualized by ethidium bromide staining A colony is selected whose PCR product was identical to the plasmid control.

Arabidopsis Transformation Arabidopsis thaliana Col-01 is transformed using the floral dip method. The selected colony is used to inoculate a 1 mL or 15 mL culture of YEP broth containing appropriate antibiotics for selection. The culture is incubated overnight at 28° with constant agitation at 220 rpm. Each culture is used to inoculate two 500 mL cultures of YEP broth containing antibiotics for selection and the new cultures are incubated overnight at 28° with constant agitation. The cells are then pelleted at approximately 8700×g for 10 minutes at room temperature, and the resulting supernatant discarded. The cell pellet is gently resuspended in 500 mL infiltration media containing: ½× Murashige and Skoog salts/Gamborg's B5 vitamins, 10% (w/v) sucrose, 0.044 μM benzylamino purine (10 μl/liter of 1 mg/mL stock in DMSO) and 300 μl/liter Silwet L-77. Plants approximately 1 month old are dipped into the media for 15 seconds, being sure to submerge the newest inflorescence. The plants are then laid down on their sides and covered (transparent or opaque) for 24 hours, washed with water, and placed upright. The plants are grown at 22°, with a 16 hr:8 hr light:dark photoperiod. Approximately 4 weeks after dipping, the seeds are harvested.

Arabidopsis Growth and Selection Freshly harvested seed is allowed to dry for at least 7 days at room temperature in the presence of desiccant. Seed is suspended in a 0.1% Agar (Sigma Chemical Co.) solution. The suspended seed is stratified at 4° for 2 days. Sunshine Mix LP5 (Sun Gro Horticulture Inc., Bellevue, Wash.) is covered with fine vermiculite and sub-irrigated with Hoagland's solution until wet. The soil mix is allowed to drain for 24 hours. Stratified seed is sown onto the vermiculite and covered with humidity domes (KORD Products, Bramalea, Ontario, Canada) for 7 days. Seeds are germinated and plants are grown in a Conviron (models CMP4030 and CMP3244, Controlled Environments Limited, Winnipeg, Manitoba, Canada) under long day conditions (16 hr light/8 hr dark) at a light intensity of 120-150 μm⁻²s⁻¹ under constant temperature)(22° and humidity (40-50%). Plants are initially watered with Hoagland's solution and subsequently with de-ionized (DI) water to keep the soil moist but not wet.

T1 seed is sown on 10.5″×21″ germination trays (T.O. Plastics Inc., Clearwater, Minn.) as described and grown under the conditions outlined. The domes are removed 5-6 days post sowing and plants are sprayed with a 1000× solution of Finale (5.78% glufosinate ammonium, Farnam Companies Inc., Phoenix, Ariz.). Two subsequent sprays are performed at 5-7 day intervals. Survivors (plants actively growing) are identified 7-10 days after the final spraying and transplanted into pots prepared with Sunshine mix LP5. Transplanted plants are covered with a humidity dome for 3-4 days and placed in a Conviron with the above mentioned growth conditions. Additional guidance concerning growth, transformation, and analysis of transgenic Arabidopsis is provided, for example, by Weigel and Glazebrook (2002).

### Example 3

### Transformation of Tobacco

Agrobacterium tumefaciens strain EHA 105 harboring binary plant transformation vectors containing plant-expressible Bt genes were prepared by standard methods. The base binary vector, pDAB7615, contains a DSM2 plant selectable marker gene positioned between Right and Left T-DNA border repeats. The full length and the modified Cry coding sequences (CDS), were first cloned into an intermediate plasmid whereby they were placed under the transcriptional control of the Cassaya Vein Mosaic Virus (CsVMV) promoter, and a 3′ Untranslated Region (UTR) derived from the Agrobacterium tumefaciens pTi15955 ORF24 gene. This plant-expressible Bt gene cassette was then cloned adjacent to the DSM2 gene in the binary vector by standard cloning methods, and the binary vector was subsequently introduced into Agrobacterium tumefaciens strain EHA 105.

Tobacco transformation with Agrobacterium tumefaciens strain EHA 105 isolates carrying binary plant transformation plasmids was carried out by a method similar, but not identical, to published methods (Horsch et al., 1988). To provide source tissue for the transformation, tobacco seed (Nicotiana tabacum cv. KY160) was surface sterilized and planted on the surface of TOB-medium, which is a hormone-free Murashige and Skoog medium (Murashige and Skoog, 1962) solidified with agar. Plants were grown for 6-8 weeks in a lighted incubator room at 28° to 30° and leaves were collected sterilely for use in the transformation protocol. Pieces of approximately one square centimeter were sterilely cut from these leaves, excluding the midrib. Cultures of the Agrobacterium strains grown overnight in a flask on a shaker set at 250 rpm and 28° were pelleted in a centrifuge and resuspended in sterile Murashige & Skoog salts, and adjusted to a final optical density of 0.5 at 600 nm. Leaf pieces were dipped in this bacterial suspension for approximately 30 seconds, then blotted dry on sterile paper towels and placed right side up on TOB+medium (Murashige and Skoog medium containing 1 mg/L indole acetic acid and 2.5 mg/L benzyladenine) and incubated in the dark at 28°. Two days later the leaf pieces were moved to TOB+medium containing 250 mg/L cefotaxime (Agri-Bio, North Miami, Fla.) and 5 mg/L glufosinate ammonium (active ingredient in Basta®, Bayer Crop Sciences) and incubated at 28° to 30° in the light. Leaf pieces were moved to fresh TOB+medium with Cefotaxime and Basta® twice per week for the first two weeks and once per week thereafter. Four to six weeks after the leaf pieces were treated with the bacteria, small plants arising from transformed foci were removed from this tissue preparation and planted into medium TOB-containing 250 mg/L Cefotaxime and 10 mg/L Basta® in Phytatray™ II vessels (Sigma Chemical Co.). These plantlets were grown in a lighted incubator room. After 3 weeks, stem cuttings were taken and re-rooted in the same media. Plants were ready to send out to the greenhouse after 2-3 additional weeks.

Plants were moved into the greenhouse by washing the agar from the roots, transplanting into soil in 13.75 cm2 pots, placing the pot into a sealed Ziploc® bag (SC Johnson & Son, Inc.), placing tap water into the bottom of the bag, and placing in indirect light in a 30° greenhouse for one week. After 3-7 days, the bag was opened; the plants were fertilized and allowed to grow in the open bag until the plants were greenhouse-acclimated, at which time the bag was removed. Plants were grown under ordinary warm greenhouse conditions (30°, 16 hr day, 8 hr night, minimum natural+supplemental light=500 μEm⁻²s⁻¹).

### Example 4

### Transformation of Maize

Agrobacterium transformation for generation of superbinary vectors To prepare for transformation, two different E. coli strains (both derived from the DH5α cloning strain) are grown at 37° overnight. The first strain contains a pSB11 derivative (Japan Tobacco. Tokyo, JP) (for example, a pDAB3878 derivative harboring a plant-expressible Bt coding region), and the second contains the conjugal mobilizing plasmid pRK2013. The pDAB3878 derivative plasmid contains the Bt-coding region under the transcriptional control of the maize ubiquitin) promoter and the maize PerS 3′UTR, and an AAD1 plant selectable marker gene, both positioned between Right and Left T-DNA border repeats. E. coli cells containing such a pDAB3878 derivative are grown on a petri plate containing LB agar medium (5 g Bacto Tryptone, 2.5 g Bacto Yeast Extract, 5 g NaCl, 7.5 g Agar, in 500 mL DI H₂O) containing Spectinomycin (100 μg/mL), and the pRK2013-containing strain is grown on a petri plate containing LB agar containing Kanamycin (50 μg/mL). After incubation the plates are placed at 4° to await the availability of the Agrobacterium strain.

Agrobacterium strain LBA4404 containing pSB1 (Japan Tobacco) is grown on AB medium with Streptomycin (250 μg/mL) and Tetracycline (10 μg/mL) at 28° for 3 days as set forth in the pSB 1 Manual (Japan Tobacco). After the Agrobacterium is ready, transformation plates were set up by mixing one inoculating loop of each bacteria (i.e., E. coli containing a pDAB3878 derivative or pRK2013, and LBA4404+pSB1) on a LB plate with no antibiotics. This plate is incubated at 28° overnight. After incubation 1 mL of 0.9% NaCl (4.5 g NaCl in 500 mL DI H₂O) solution is added to the mating plate and the cells are mixed into the solution. The mixture is then transferred into a labeled sterile Falcon 2059 (Becton Dickinson and Co. Franklin Lakes, N.J.) tube or equivalent. Another mL of 0.9% NaCl is added to the plate and the remaining cells are mixed into the solution. This mixture is then transferred to the same labeled tube as above.

Serial dilutions of the bacterial cells are made ranging from 10⁻¹ to 10⁻⁴ by placing 100 μL of the bacterial “stock” culture into labeled Falcon 2059 tubes and then adding 900 μL of 0.9% NaCl. To ensure selection, 100 μL of the dilutions are then plated onto separate plates containing AB medium with Spectinomycin (100 μg/mL), Streptomycin (250 μg/mL), and Tetracycline (10 μg/mL) and incubated at 28° for 4 days. The colonies are then “patched” onto AB+Spec/Strep/Tet plates as well as lactose medium (0.5 g Yeast Extract, 5 g D-lactose monohydrate, 7.5 g Agar, in 500 mL DI H₂O) plates and placed in the incubator at 28° for 2 days.

A Keto-lactose test is performed on the colonies on the lactose media by flooding the plate with Benedict's solution (86.5 g Sodium Citrate monobasic, 50 g Na₂CO₃, 9 g CuSO₄.5 H₂O, in 500 ml, of DI H₂O) and allowing the Agrobacterium colonies to turn yellow. Any colonies that are yellow (positive for Agrobacterium) are then picked from the patch plate and streaked for single colony isolation on AB+Spec/Strep/Tet plates at 28° for 2 days.

One colony per plate is picked for a second round of single colony isolations on AB+Spec/Strep/Tet media and this is repeated for a total of three rounds of single colony isolations. After the single-colony isolations, plasmid DNA is prepared from each isolate for transfer into E. coli to facilitate plasmid structure validation. One colony per plate is picked and used to inoculate separate 3 mL YEP (5 g Yeast Extract, 5 g Peptone, 2.5 g NaCl, in 500 mL DI H₂O) liquid cultures containing Spectinomycin (100 μg/mL), Streptomycin (250 μg/mL), and Tetracycline (10 μg/mL). These liquid cultures are then grown overnight at 28° in a rotary drum incubator at 200 rpm. Validation cultures are then started by transferring 2 mL of the inoculation cultures to 250 mL disposable flasks containing 75 mL of YEP+Spec/Strep/Tet. These are then grown overnight at 28° while shaking at 200 rpm. Following the Qiagen® protocol, Hi-Speed maxi-preps are then performed on the bacterial cultures to produce plasmid DNA. 500 μL of the eluted DNA is then transferred to 2 clean, labeled 1.5 mL tubes and the Edge BioSystems (Gaithersburg, Md.) Quick-Precip Plus® protocol is followed.

After the precipitation the plasmid DNA is resuspended in a total volume of 100 μL TE (10 mM Tris HCl, pH 8.0; 1 mM EDTA). 5 μL of plasmid DNA is added to 50 μL of chemically competent DH5a (Invitrogen) E. coli cells and gently mixed. This mixture is then transferred to chilled and labeled Falcon 2059 tubes. The reaction is incubated on ice for 30 minutes and then heat shocked at 42° for 45 seconds. The reaction is placed back into the ice for 2 minutes and then 450 μL of SOC medium (Invitrogen) s added to the tubes. The reaction is then incubated at 37° for 1 hour, shaking at 200 rpm. The cells are then plated onto LB+Spec/Tet (using 50 μL and 100 μL of cells) and incubated at 37° overnight.

Three or four colonies per plate are picked and used to inoculate separate 3 mL LB liquid cultures containing Spectinomycin (100 μg/mL), and Tetracycline (10 μg/mL). These liquid cultures are then grown overnight at 37° in a drum incubator at 200 rpm. Following the Qiagen® protocol, mini-preps are then performed on the bacterial cultures to produce plasmid DNA. 5 μL of plasmid DNA is then digested in separate reactions using Hind III and Sal I, or other appropriate enzymes (NEB) at 37° for 1 hour before analysis on a 1% agarose (Cambrex Bio Science Rockland, Inc., Rockland, Me.) gel. The plasmid lineage of the E. coli culture that shows the correct banding pattern is then used to track back to the Agrobacterium isolate that harbored the correct plasmid. That Agrobacterium isolate is grown up and used to create glycerol stocks by adding 500 μL of culture to 500 μL of sterile glycerol (Sigma Chemical Co.) and inverting to mix. The mixture is then frozen on dry ice and stored at −80° until needed.

Agrobacterium-Mediated Transformation of Maize Seeds from a High II F1 cross (Armstrong et al., 1991) are planted into 5-gallon-pots containing a mixture of 95% Metro-Mix 360 soilless growing medium (Sun Gro Horticulture, Bellevue, Wash.) and 5% clay/loam soil. The plants are grown in a greenhouse using a combination of high pressure sodium and metal halide lamps with a 16 hr:8 hr light:dark photoperiod. For obtaining immature F2 embryos for transformation, controlled sib-pollinations are performed. Immature embryos are isolated at 8-10 days post-pollination when embryos are approximately 1.0 to 2.0 mm in size.

Infection and cocultivation Maize ears are surface sterilized by scrubbing with liquid soap, immersing in 70% ethanol for 2 minutes, and then immersing in 20% commercial bleach (0.1% sodium hypochlorite) for 30 minutes before being rinsed with sterile water. The Agrobacterium suspension is prepared by transferring for 2 loops of bacteria grown on YEP medium with 15 g/L Bacto agar containing 100 mg/L Spectinomycin, 10 mg/L Tetracycline, and 250 mg/L Streptomycin at 28° for 2-3 days into 5 mL of liquid infection medium (LS Basal Medium (Linsmaier and Skoog, 1965), N6 vitamins (Chu et al., 1975), 1.5 mg/L 2,4-D, 68.5 g/L sucrose, 36.0 g/L glucose, 6 mM L-proline, pH 5.2) containing 100 μM acetosyringone. The solution is vortexed until a uniform suspension is achieved, and the concentration is adjusted to a final density of 200 Klett units, using a Klett-Summerson colorimeter with a purple filter. Immature embryos are isolated directly into a micro centrifuge tube containing 2 mL of the infection medium. The medium is removed and replaced with 1 mL of the Agrobacterium solution with a density of 200 Klett units. The Agrobacterium and embryo solution is incubated for 5 minutes at room temperature and then transferred to co-cultivation medium (LS Basal Medium, N6 vitamins, 1.5 mg/L 2,4-D, 30.0 g/L sucrose, 6 mM L-proline, 0.85 mg/L AgNO3,1, 100 μM acetosyringone, 3.0 g/L Gellan gum, pH 5.8) for 5 days at 25° under dark conditions.

After co-cultivation, the embryos are transferred to selective media after which transformed isolates are obtained over the course of approximately 8 weeks. For selection, an LS based medium (LS Basal medium, N6 vitamins, 1.5 mg/L 2,4-D, 0.5 g/L MES, 30.0 g/L sucrose, 6 mM L-proline, 1.0 mg/L AgNO3, 250 mg/L Cephotaxime, 2.5 g/L Gellan gum, pH 5.7) is used with Bialaphos. The embryos are transferred to selection media containing 3 mg/L Bialaphos until embryogenic isolates are obtained. Any recovered isolates are bulked up by transferring to fresh selection medium at 2-week intervals for regeneration and further analysis.

Regeneration and seed production For regeneration, the cultures are transferred to “28” induction medium (MS salts and vitamins, 30 g/L sucrose, 5 mg/L benzylaminopurine, 0.25 mg/L 2,4-D, 3 mg/liter Bialaphos, 250 mg/L Cephotaxime, 2.5 g/L Gellan gum, pH 5.7) for 1 week under low-light conditions (14 μEm⁻²s⁻¹) then 1 week under high-light conditions (approximately 89 μEm⁻²s⁻¹). Tissues are subsequently transferred to “36” regeneration medium (same as induction medium except lacking plant growth regulators). When plantlets grow to 3-5 cm in length, they are transferred to glass culture tubes containing SHGA medium (Schenk and Hildebrandt salts and vitamins (Schenk and Hildebrandt, 1972), 1.0 g/L myo-inositol, 10 g/L sucrose and 2.0 g/L Gellan gum, pH 5.8) to allow for further growth and development of the shoot and roots. Plants are transplanted to the same soil mixture as described earlier herein and grown to flowering in the greenhouse. Controlled pollinations for seed production are conducted.

### Example 5

### Nematode Bioassay of Transgenic Plants Expressing Cry Toxins

T1 transgenic plants containing the Cry toxin genes were characterized with regard to expression levels and intactness of the transgenic protein. Following characterization, the plants are challenged with plant pathogenic nematodes utilizing established methods (Urwin et al., 2003; McLean et al., 2007; Goggin et al., 2006). Root damage, feeding sites and nematode egg production are quantified and compared.

Specifically, T0 transgenic tobacco plants transformed to contain plant-expressible Cry toxin genes of this invention were bioassayed for reduced nematode reproduction. Currently, data reported herein was obtained from plants expressing (individually) SEQ ID NOs:5 or 12. Transgenic, herbicide-selected tissue culture plants were transplanted when they were approximately three inches tall. Non-transgenic control plants were taken from tissue culture without any selective agent. Plants were transplanted into approximately 200 cubic centimeters of potting mix (80% sand, 20% peat based potting mix) in 8 cm round pots and grown 1-2 weeks prior to inoculation. Three leaf discs (˜1 cm) were taken from a middle leaf of each plant for immunoblot analysis prior to inoculation. The three leaf discs were ground and suspended in 200 μL of SDS-PAGE loading buffer. The proteins were resolved on 5-20% gradient gels, electroblotted onto PVDF membrane, and probed with the appropriate antibody at dilutions ranging from 1:1000 to 1:2000. Immunoblot detection was performed using an alkaline phosphatase conjugated secondary antibody and NBT-BCIP detection reagent by standard methods (Coligan et al., 2007, and updates).

All plants were inoculated with 1000 Meloidogyne incognita J2 stage juveniles applied near the base of each plant in 1 mL of water. Plants were incubated in a growth room with 14 hr:10 hr (light:dark) photoperiod and an average temperature of 22° for the duration of the experiment (typically 50 to 60 days post inoculation). Eggs were harvested from the root mass of each plant using a standard bleach extraction procedure.

Briefly, plants were harvested and the roots were photographed after lightly rinsing in water to remove loosely attached soil. A subjective “galling” index was estimated and recorded for each sample. Roots were removed and weighed prior to being chopped and suspended in 10% bleach in a 1 liter beaker. All plants were treated with rooting hormone and repotted after root harvest for seed production. Chopped roots were stirred in 10% bleach for 10 min using a paddle stirrer. The root suspension was then passed through a strainer to remove roots and then into nested sieves of 74 μm and 30 μm to harvest the eggs. The sieves were extensively rinsed with water and the eggs were recovered from the 30 μm sieve by rinsing with approximately 10 mL of water into a 15 mL conical screw cap tube. Dilution series were prepared for each sample in 24 well microtitre plates and each well was photographed using an Olympus IX51 inverted microscope equipped with a digital camera. Dilutions with a suitable number of eggs were counted for each sample. Egg counts were converted to eggs per gram fresh root weight (eggs/gmFW) and tabulated.

As a preliminary indication of the effectiveness of the subject Cry toxins, nematode challenges were performed on both immunoblot-positive and immunoblot-negative T0 transgenic tobacco plants. The number of eggs/gmFW of roots of non transformed (i.e. wild-type) plants was used to compare to the eggs/gmFW counts for transgenic plants. A range of eggs/gmFW counts was seen for the transgenic plants. Isolates were recovered that yielded below 1 standard deviation from the mean eggs/gmFW counts of nontransformed plants. As may be expected by one familiar with analyses of T0 transgenic plants, some of the T0 plants had egg counts higher than or no different from the numbers obtained from nontransformed control plants.

### REFERENCES

An, G., Watson, B. D., Stachel, S., Gordon, M. P., Nester, E. W. (1985) New cloning vehicles for transformation of higher plants. EMBO J. 4:277-284.
Armstrong, C. L., Green, C. E., Phillips, R. L. (1991) Development and availability of germplasm with high Type II culture formation response. Maize Coop. News Lett. 65:92-93.
Ausubel et al., eds. (1995) Current Protocols in Molecular Biology, (Greene Publishing and Wiley-Interscience, New York)
Betz, F. S., Hammond, B. G., Fuchs, R. L. (2000) Safety and advantages of Bacillus thuringiensis-protected plants to control insect pests. Regul. Toxicol. Pharmacol. 32:156-173.
Bone, L. W., Bottjer, K. P., Gill, S. S. (1985) Trichostrongylus colubriformis: egg lethality due to Bacillus thuringiensis crystal toxin. Exper. Parasitol. 60:314-322.
Bone, L. W., Bottjer, K. P., Gill, S. S. (1987) Alteration of Trichostrongylus colubriformis egg permeability by Bacillus thuringiensis israelensis toxin. J. Parasitol. 73:295-299.
Bone, L W., Bottjer, K. P, Gill, S. S. (1988) Factors affecting the larvicidal activity of Bacillus thuringiensis israelensis toxin for Trichostrongylus colubriformis (Nematoda). J. Invert. Pathol. 52:102-107.
Bottjer, K. P., Bone, L. W., Gill, S. S. (1985) Nematoda: susceptibility of the egg to Bacillus thuringiensis toxins. Exper. Parasitol. 60:239-244.
Bravo, A., Gill, S. S., Soberon, M. (2007) Mode of action of Bacillus thuringiensis Cry and Cyt toxins and their potential for insect control. Toxicon. 49:423-435.
Caruthers, M. H., Kierzek, R., Tang, J. Y. (1987) Synthesis of oligonucleotides using the phosphoramidite method. Bioactive Molecules (Biophosphates Their Analogues) 3:3-21
Chitwood, D. J. (2003) Nematicides. In J. R. Plimmer, ed. Encyclopedia of Agrochemicals. Vol. 3. Published by John Wiley & Sons, New York, N.Y. pp. 1104-1115.
Chu, C. C., Wang, C. C., Sun, C. S., Hsu, C., Yin, K. C., Chu, C. Y., Bi, F. Y. (1975) Establishment of an efficient medium for another culture of rice through comparative experiments on the nitrogen sources. Sci. Sinica 18:659-668.
Coligan, J. E., et al., eds. Current Protocols in Immunology (2007), John Wiley & Sons, Inc., NJ
Fraley, R. T., Rogers, S. G., Horsch, R. B. (1986) Genetic transformation in higher plants. Crit. Rev. Plant Sci. 4:1-46.
Goggin, F. L., Jia, L., Shah, G., Williamson, V. M., Ullman, D. E. (2006.) The tomato Mi-1.2 herbivore resistance gene functions to confer nematode resistance but not aphid resistance in eggplant. Molec. Plant-Microbe Interact. 19: 383-388.
Hoekema, A. (1985) The Binary Plant Vector System: New approach to genetic engineering of plants via Agrobacterium tumefaciens. Published by Proefsciar., Rijksuniv. Leiden, Albasserdam, Durkkerij Kanters B.V., Chapter 5.96 p.
Holsters, M., De Waele, D., Depicker, A., Messens, E., Van Montagu, M., Schell, J. (1978) Transfection and transformation of Agrobacterium tumefaciens. Molec. Gen. Genet. 163:181-187.
Horsch, R. B, Fry, J., Hoffmann, N., Neidermeyer, J., Rogers, S. G., Fraley R. T. (1988) Leaf disc transformation. In Plant Molecular Biology Manual, S. B. Gelvin, R. A. Schilperoort and D. P. S. Verma, eds., Published by Kluwer Academic Publishers, Boston. p. 1-9.
Li, X.-Q., Wei, J.-Z., Tan, A., Aroian, R. V. (2007) Resistance to root-knot nematode in tomato roots expressing a nematicidal Bacillus thuringiensis crystal protein. Plant Biotech. J. 5:455-464.
Linsmaier, E. M., Skoog, F. (1965) Organic growth factor requirements of tobacco tissue cultures. Physiol. Plant. 18:100-127.
McLean, M. D, Hoover, G. J., Bancroft, B., Makhmoudova, A., Clark, S. M., Welacky, T., Simmonds, D. H., Shelp, B. J. (2007) Identification of the full-length Hs1^{pro-1} coding sequence and preliminary evaluation of soybean cyst nematode resistance in soybean transformed with Hs1^{pro-1} cDNA. Can. J. Bot. 85:437-441.
Murashige, T., Skoog, F. (1962) Revised medium for rapid growth and bioassays with tobacco tissue cultures. Physiol. Plant. 15:473-497.
Ristaino, J. B., Thomas, W. (1997) Agriculture, methyl bromide, and the ozone hole: can we fill the gaps? Plant Dis. 81:965-977.
Sambrook, J., Fritsch, E. F. & Maniatis, T. (1989) Molecular Cloning: A Laboratory Manual (2nd ed., Cold Spring Harbor Laboratory Press, Plainview, N.Y.)
Sasser, J. N., Freckman, D. W. (1987) A world perspective on nematology: the role of the society. In Vistas on Nematology. J.A. Veech and D. W. Dickson, eds. Published by Society of Nematologists, Hyattsville, Md., pp. 7-14.
Schenk, R. U., Hildebrandt, A. C. (1972) Medium and techniques for induction and growth of monocotyledonous and dicotyledonous plant cell cultures. Can. J. Bot. 50:199-204.
Schnepf, E., Crickmore, N., Van Rie, J., Lereclus, D., Baum, J., Feitelson, J., Zeigler, D. R., Dean, D. H. (1998) Bacillus thuringiensis and its pesticidal crystal proteins. Microbiol. Mol. Biol. Rev. 62:775-806.
Stewart, L., Burgin, A. B., (2005) Whole gene synthesis: a gene-o-matic future. Frontiers Drug Design Disc. 1:297-341.
Urwin, P. E., Green, J., Atkinson, H. J. (2003) Expression of a plant cystatin confers partial resistance to Globodera, full resistance is achieved by pyramiding a cystatin with natural resistance. Molec. Breed. 12:263-269.
Wei, J.-Z., Hale, K., Carta, L., Platzer, E., Wong, C., Fang, S.-C., Aroian, R. V. (2003) Bacillus thuringiensis crystal proteins that target nematodes. Proc. Natl. Acad. Sci. 100:2760-2765.
Weigel, D., Glazebrook, J. [eds.] (2002) Arabidopsis: A Laboratory Manual. Cold Spring Harbor Press, Cold Spring Harbor, N.Y., 354 pages.

### PATENTS CITED

U.S. Pat. No. 5,380,831
U.S. Pat. No. 5,589,382
U.S. Pat. No. 5,616,495
U.S. Pat. No. 5,753,492
U.S. Pat. No. 6,218,188
U.S. Pat. No. 6,632,792
U.S. Pat. No. 6,673,990
U.S. Pat. No. 7,122,516

1. A transgenic plant that is resistant to damage by a nematode, wherein said resistance is due to expression of a polynucleotide that encodes a Cry6 protein that has toxin activity against said nematode.

2. The plant of claim 1 wherein said Cry protein is a modified Bacillus thuringiensis Cry protein, and said protein is truncated at the N terminus and/or at the C terminus, as compared to a corresponding full-length protein.

3. The plant of claim 2 wherein said protein is truncated at the N terminus, as compared to the corresponding full-length protein.

4. The plant of claim 2 wherein said protein is truncated at the C terminus, as compared to the corresponding full-length protein.

5. The plant of claim 2 wherein said protein is truncated at the N terminus and at the C terminus, as compared to the corresponding full-length protein.

6. The plant of claim 1 wherein said nematode is selected from the group consisting of root knot nematode (Meloidogyne incognita) and soybean cyst nematode (Heterodera glycines).

7. The plant of claim 1 wherein said polynucleotide is operably linked to a root-specific promoter.

8. The plant of claim 1 wherein said Cry protein is a Cry6A protein.

9. The plant of claim 1, said polynucleotide comprising codon usage for increased expression in a plant.

10. A polynucleotide that encodes a modified Bacillus thuringiensis Cry6A protein having toxin activity against a nematode wherein said protein is truncated at the N terminus and/or at the C terminus, as compared to a corresponding full-length protein.

11. A modified protein encoded by the polynucleotide of claim 10.

12. The polynucleotide of claim 10 wherein said protein is truncated at the N terminus, as compared to the corresponding full-length protein.

13. The polynucleotide of claim 10 wherein said protein is truncated at the C terminus, as compared to the corresponding full-length protein.

14. The polynucleotide of claim 10 wherein said protein is truncated at the N terminus and at the C terminus, as compared to the corresponding full-length protein.

15. The polynucleotide of claim 10, said polynucleotide comprising codon usage for increased expression in a plant.

16. A polynucleotide that comprises a sequence selected from the group consisting of SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, and SEQ ID NO: 13.

17. A protein that comprises a sequence selected from the group consisting of SEQ ID NO: 3, SEQ ID NO: 6, SEQ ID NO: 9, SEQ ID NO: 12, and SEQ ID NO:

18. A plant cell comprising a polynucleotide of claim 10.

19. A plant comprising a plurality of cells of claim 18.

20. A plant cell that produces a protein of claim 11.

21. A plant that produces a protein of claim 11.

22. The polynucleotide of claim 10 wherein said nematode is selected from the group consisting of root knot nematode (Meloidogyne incognita) and soybean cyst nematode (Heterodera glycines).

23. A method of inhibiting a nematode, said method comprising providing to said nematode a protein of claim 11 and/or 17 for ingestion.

24. The method of claim 23 wherein said protein is produced by and is present in a plant.

25. A plant cell comprising a polynucleotide of claim 16.

26. A plant cell that produces a protein of claim 17.

27. A plant that produces a protein of claim 17.

28. The polynucleotide of claim 16 wherein said nematode is selected from the group consisting of root knot nematode (Meloidogyne incognita) and soybean cyst nematode (Heterodera glycines).

29. A method of inhibiting a nematode, said method comprising providing to said nematode a protein of claim 17 for ingestion.
